(19) United States
(12) Patent Application Publication
(10) Pub. No.: US 2023/0279344 A1
(43) Pub. Date: Sep. 7, 2023

(54) AIR-LOADED GAS VESICLE NANOPARTICLES FOR PROMOTING CELL GROWTH IN 3D BIOPRINTED TISSUE CONSTRUCTS

(71) Applicant: KING ABDULLAH UNIVERSITY OF SCIENCE AND TECHNOLOGY, Thuwal (SA)

(72) Inventors: Salwa Alshehri, Thuwal (SA); Charlotte Hauser, Thuwal (SA); Ram Karan, Thuwal (SA); Magnus Rueping, Thuwal (SA)

(21) Appl. No.: 18/024,072

(22) PCT Filed: Sep. 1, 2021
(86) PCT No.: PCT/IB2021/057973

§ 371 Date: Mar. 1, 2023

Related U.S. Application Data
(60) Provisional application No. 63/073,516, filed on Sep. 2, 2020.

(51) Int. Cl. C12N 5/00; C07K 7/06; C12N 15/74; C09D 11/04

(52) U.S. Cl. CPC C12N 5/0062; C07K 7/06; C12N 15/74; C09D 11/04

(57) ABSTRACT

The present disclosure relates to a method of high yielding production of gas vesicle nanoparticles (GVNPs) and genetic tools used for high-yielding GVNPs production. The present disclosure further relates to a method of creating 3D tissue constructs with improved cell viability and proliferation and the resulting 3D tissue constructs. The GVNPs can promote cell growth and proliferation in 3D constructs and are suitable bioinks components for a bioprinter to build 3D structures through 3D printing as well as other applications.

### CROSS-REFERENCE TO RELATED APPLICATIONS

This application claims benefit of priority of U.S. Provisional Patent Application No. 63/073,516 entitled, “AIR-LOADED GAS VESICLE NANOPARTICLES PROMOTE CELL GROWTH IN 3D BIOPRINTED TISSUE CONSTRUCTS” filed Sep. 2, 2020. The entire contents and disclosures of these patent applications are incorporated herein by reference in their entirety.

### REFERENCE TO A “SEQUENCE LISTING”

The present application includes a Sequence Listing which has been submitted electronically in an ASCII text format. This Sequence Listing is named 114147-23830US01_sequence listing.TXT was created on Aug. 27, 2021, is 1,418 bytes in size and is hereby incorporated by reference in its entirety.

### BACKGROUND

### Field of the Invention

The present disclosure relates to a method of high yielding production of gas vesicle nanoparticles (GVNPs) and tools used for high-yielding GVNPs production. The present disclosure further relates to a method of creating 3D tissue constructs with improved cell viability and proliferation and the resulting 3D tissue constructs.

### Background of the Invention

Human organs may be defined by their complex, functional organization of diverse, specialized cells and tissues¹. The rising incidence rates of disease and injury leading to organ failure have led to increased demand for tissue and organ transplants. This growing demand has given rise to tissue engineering, whereby scaffolds, cells, and biologically active molecules are combined to assemble functional constructs. The goal is to develop constructs capable of restoring, improving, or maintaining damaged tissues and organs². Of the many methods to generate these functional three-dimensional structures, 3D-bioprinting is most promising due to its ability to produce complex, scalable tissue constructs. The printing of these cellularized structures through the precise layering of cell-laden bioinks may be accomplished using inkjet-based, extrusion, stereolithography, or laser-assisted printing systems³⁻⁵. While each printer has its advantages over the others, many challenges remain to face before this technology can be translated into clinical practice, regardless of the printer setup⁶.

Significant issues with 3D bioprinting include identifying suitable bioinks and ensuring adequate nutrient supply to the cells⁷⁻¹². While a great deal of effort has been focused on developing novel biomaterial candidates, it has been much harder to find a solution to address the diffusion barrier associated with the printed construct. The diffusion barrier is an inherent result of the nature of the bioink scaffolding material, which hinders the migration of nutrients, ranging from growth factors to oxygen, to the center. This diffusion barrier must be overcome as delivering these nutrients ensures the cells' survival, proliferation, and differentiation.

As such, oxygen-releasing biomaterials have been explored as a means of promoting its diffusion throughout the construct. Most of the oxygen-releasing biomaterials developed so far involve scaffolds integrated with peroxides and fluorinated compounds in the form of liquids or solid micro- and nanoparticles^{13, 14}. Although early reports of these materials are promising, they all require the insertion of materials that do not occur naturally within the human body¹⁵⁻¹⁷. This introduces additional uncertainty about the fate of these materials as they lack the inherent biodegradability of bio-molecules, such as proteins.

Due to the limitations of the oxygen-releasing biomaterials, there exists a need for a new class of biomaterials that can facilitate nutrient migration within the tissue constructs.

Furthermore, haloarchaeal gas vesicle nanoparticles (GVNPs) are attractive for biotechnological and biomedical applications and have already found use as drug delivery systems and contrast agents for ultrasound and magnetic resonance imaging²²⁻²⁷. While the biotechnological tools for the expression of GVNPs are established, production so far suffers from the slow growth rates, the inconsistent induction system, and genetic instability of the Halobacterium expression host.

Therefore, a method of fast and efficient production of GVNPs is also needed.

In addition, it is also desirable to provice a biocompatible compound that is capable of forming a hydrogel that meets at least some of the above requirements to a higher extent than currently available hydrogels and that is not restricted by the above mentioned limination.

### SUMMARY

According to a first broad aspect the present disclosure provides a 3-dimensional construct comprising ultrashort peptide scaffold; gas vesicle; and at least one mammalian cells.

According to a second broad aspect the present disclosure provides a method of high-yielding production of gas vesicle comprising amplifying a gas vesicle operon from the genome of a gas vesicle operon containing bacterium; cloning the gas vesicle operon into an expression plasmid; transforming the gas vesicle operon containing expression plasmid into Haloferax volcanii; culturing the Haloferax volcanii transformed with the gas vesicle operon containing expression plasmid; lysing the Haloferax volcanii transformed with the gas vesicle operon containing expression plasmid; and collecting the gas vesicle.

According to a third broad aspect the present disclosure provides a method of creating 3-dimensional construct comprising dissolving at least one ultrashort peptide to form a peptide solution; mixing mammalian cells with the peptide solution; dissolving the gas vesicle to form a gas vesicle solution; adding the gas vesicle solution to the mammalian cells containing peptide solution; and building 3-dimensional construct using the peptide solution containing both gas vesicle and mammalian cells; wherein the ultrashort peptide is dissolved in water or buffer solution.

Other aspects and features of the present disclosure will become apparent to those skilled in the art upon review of the following description of specific embodiments of the invention in conjunction with the accompanying figures.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a photo showing H. volcanii cells grown in liquid culture according to an embodiment of the present disclosure.

FIG. 2 is a photo showing the pure white gas vesicles according to an embodiment of the present disclosure.

FIG. 3 is a transmission electron microscopic photo showing the intact gas vesicles according to an embodiment of the present disclosure.

FIG. 4 is a transmission electron microscopic photo showing an individual intact gas vesicle according to an embodiment of the present disclosure.

FIG. 5 is a scanning electron microscopic photo showing the network of IK₆ hydrogels according to an embodiment of the present disclosure.

FIG. 6 is a bright field microscopic photo showing the HEK cells in 2D culture after one day of culture at varying GVNP concentrations according to an embodiment of the present disclosure.

FIG. 7 is a fluorescent microscopic photo showing the HEK cells in 2D culture with or without GVNPs according to an embodiment of the present disclosure.

FIG. 8 is a bright field microscopic photo showing the HEK cells in 3D culture after one day of culture at varying GVNP concentrations according to an exemplary embodiment of the present disclosure.

FIG. 9 is a graph showing the cell proliferation rate of HEK cells in 3D construct at different time points according to an exemplary embodiment of the present disclosure.

FIG. 10 is an image showing a 1 cm cylinder printed with IK₆ peptide (SEQ ID No. 1) according to an exemplary embodiment of the present disclosure.

FIG. 11 is a scanning electron microscopic photo showing the morphological characterization of the printed peptide scaffold with GVNPs according to an exemplary embodiment of the present disclosure.

FIG. 12 is a confocal image showing the engineered sfGFP gas vesicles within the 3D printed sample according to an exemplary embodiment of the present disclosure.

FIG. 13 is live/dead staining images showing the viability of printed cells with and without GVNPs at days 1, 4, and 7 after printing according to an exemplary embodiment of the present disclosure.

FIG. 14 is a graph illustrating the quantitative analysis results of cell viability according to an exemplary embodiment of the present disclosure.

FIG. 15 a fluorescence confocal microscopy showing the proliferation of cells in hydrogels after printing according to an exemplary embodiment of the present disclosure.

FIG. 16 an electron microscope images showing printed cells with and without GVNPs according to an exemplary embodiment of the present disclosure.

FIG. 17 an image showing the 3D bioprinter setup according to an exemplary embodiment of the present disclosure.

FIG. 18 is a graph showing a preview of the gcode file of the printed structure with dimensions measuring 10 mm×10 mm×1.5 mm according to an embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “amphiphilic” or “amphiphilicity” refers to being a compound consisting of molecules having a water-soluble group at one end and a water-insoluble group at the other end.

For purposes of the present disclosure, the term “bioinks” as used herein means materials used to produce engineered/artificial live tissue, cellular grafts and organ substitutes (organoids) using 3D printing. In the present disclosure, these bioinks are mostly composed of hydrogel or organogel with cellular components embedded.

For purposes of the present disclosure, the term “gel”, “nanogel” “hydrogel” and “organogel” are used interchangeably. These terms refer to a is a network of polymer chains, entrapping water or other aqueous solutions, such as physiological buffers, of over 99% by weight. In an embodiment of the present disclosure, the polymer chains may be a peptide with repetitive sequences. If the self-assembly of the ultrashort peptides occurs in aqueous solution, hydrogels are formed. If organic solvents are used, organogels are formed.

For purposes of the present disclosure, the term “operon” refers to is a functioning unit of DNA containing a cluster of genes under the control of a single promoter. The genes are transcribed together into an mRNA strand and translated into at least one gene product.

For purposes of the present disclosure, the term “P/S” refers to penicillin-streptomycin solution.

For purposes of the present disclosure, the term “PBS” refers to a buffer solution commonly used in biological research, which is an abbreviation of phosphate-buffered saline. It is a water-based salt solution, helping to maintain a constant pH, as well as osmolarity and ion concentrations to match those of most cells. In some embodiments, PBS may include a water-based salt solution containing disodium hydrogen phosphate, sodium chloride and, in some formulations, potassium chloride and potassium dihydrogen phosphate.

For purposes of the present disclosure, the term “printability” refers to the suitability of peptide for 3D printing. In particular, it refers to the suitable speed of self-assembly at certain concentration, and viscosity. The speed of forming gel and viscosity need to be high enough so that a structure with certain height can be printed without collapsing. On the other hand, the speed and viscosity need to be low enough so that the peptide will not clog the nozzle of bioprinters.

For purposes of the present disclosure, the term “scaffolds” as used herein means the supramolecular network structures made from self-assembling ultra-short peptide or other polymer materials in the bioinks that provide support for the cellular components.

For purposes of the present disclosure, the term “structure fidelity” refers to the ability of 3D constructs to maintain its shape and internal structure over time.

For purposes of the present disclosure, the term “ultra-short peptide” refers to a sequence containing 3-7 amino acids. The peptides according an aspect of the present disclosure are also particularly useful for formulating aqueous or other solvent compositions, herein also sometimes referred to as “inks” or “bioinks” when mixed with cellular components, which may be used as inks for printing structures and as bioinks for printing cellular or tissue structures, in particular 3D structures. Such printed structures make use of the gelation properties of the peptides according to features of the present disclosure.

For purposes of the present disclosure, the terms “biocompatible” (which also can be referred to as “tissue compatible”) and “biocompatibility”, as used herein, refer to the property of a hydrogel that produces little if any adverse biological response when used in vivo.

For purposes of the present disclosure, the terms “v/v”, “v/v %” and “% v/v” are used interchangeably. These terms refer to Volume concentration of a solution.

For purposes of the present disclosure, the terms “w/v”, “w/v %” and “% w/v” are used interchangeably. These terms refer to Mass concentration of a solution, which is expressed as weight per volume.

For purposes of the present disclosure, the terms “homologously distributed” and “homologous distribution” refers to that the density or concentration of GVNPs, cells or other components in the 3D construct is about the same in different parts of the 3D construct.

### DESCRIPTION

3D bioprinting has emerged as a promising method for the engineering of tissues and organs. Still, it faces challenges in its widespread use due to issues with the development of bioink materials and the nutrient diffusion barrier inherent to these scaffold materials. Disclosed embodiments introduce a method to promote oxygen diffusion throughout the printed constructs using genetically encoded gas vesicles derived from haloarchaea. These hollow nanostructures are comprised of a protein shell that allows gases to permeate freely while excluding the water flow. After printing cells with gas vesicles of various concentrations, the cells are observed to have increased activity and proliferation. These results suggest that air-filled gas vesicles can help overcome the diffusion barrier throughout the 3D bioprinted constructs by increasing oxygen availability to cells within the center of the construct. The biodegradable nature of the gas vesicle proteins combined with the disclosed promising results permits their potential use as oxygen-promoting materials in biological samples.

In one embodiment, gas vesicle nanoparticles (GVNPs) are used to promote the oxygen diffusion within 3D tissue constructs.

Gas vesicles are hollow gas-filled proteinaceous intracellular organelles common to many species of bacteria and archaea 18. In nature, gas vesicles promote floatation and the availability of oxygen in the microbial cell¹⁹. These cylindrical- or spindle-shaped organelles have conical ends and vary in size depending on the organism^{20, 21}. Over the years, gas vesicle nanoparticles have drawn interest in biotechnological and biomedical applications. This includes traditional nanoparticle applications as drug delivery systems and other applications based on their unique physical properties. It has been discovered that gas vesicles possess sound scattering properties and have the ability to produce harmonic ultrasound signals. One pioneering use of GVNPs is as novel contrast agents and molecular sensors for ultrasound and magnetic resonance besides others²²⁻²⁷.

In one embodiment, a new, efficient system for haloarchaeal Haloferax volcanii gas vesicle expression is developed. The system utilizes a combination of attributes, such as moderate temperature, fast and elevated cell growth, no requirement for antibiotics), to facilitate cheaper and faster GVNP production at yields high enough to be suitable for bioreactor scale^{22, 28}.

In one embodiment, gas vesicles are printed homogeneously throughout the 3D tissue constructs, to improve cell growth by promoting the diffusion of oxygen.

In one embodiment, the bioprinting of gas vesicles is conducted with a 3D bioprinting system designed for ultra-short peptide hydrogels²⁹⁻³³, wherein the printability of peptide bioinks and biocompatibility with various cell types was confirmed to be high^{34, 35}.

Gas vesicles have been reported to promote cell activity in cell culture³⁶. In one embodiment, gas vesicles are used to promote cell activity, in order to attain better outcomes in 3D bioprinting.

In one embodiment, the biocompatibility of the gas vesicles was tested with human embryonic kidney cells in both 2D and 3D cell cultures.

In one embodiment, the printability of the gas vesicles is sufficient to ensure that the nanoparticles are capable of withstanding the shear stress involved in the printing process.

In one embodiment, the bioink and scaffolding material used throughout this study was IK₆ self-assembling peptide (SEQ ID No. 1).

In one embodiment, the 3D printing of gas vesicles may positively affect cell activity for up to seven days when printed together with cells, as evidenced by high cell viability and healthy morphology of cells printed with gas vesicles.

### GVNPs Characterization

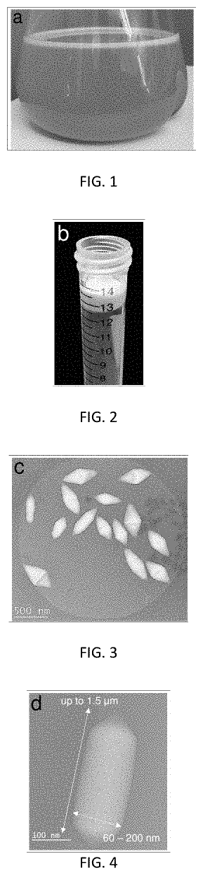

In one embodiment, expression of the gas vesicle operon in H. volcanii leds to spindle- or cylinder-shaped gas vesicles with conical tips, diameters of 60-200 nm, and lengths of up to 1.5 μm. FIG. 1 shows the gas vesicle expressiong H. volcanii cells grown in liquid culture. As shown in FIG. 1, buoyant cells floated at the top, after bing left to stand. The pure white gas vesicles floating at the top is shown in FIG. 2.

In one embodiment, the shape and size of intact gas vesicles nanoparticles are determined by transmission electron microscopy (FEI Titan CT microscope) equipped with a 4 k×4 k CCD camera (Gatan®, Pleasanton, Calif., USA). FIG. 3 shows a transmission electron microscopic (TEM) photo of the spindle-shaped intact gas vesicles. In one exemplary embodiment, FIG. 4 shows an individual intact gas vesicle, with its size indicated. As shown in FIG. 4, the intact gas vesicle has a length of up to 1.5 μm and width of about 60-200 nm.

In one embodiment, the TEM imaging of the cultured gas vesicle nanoparticles showed that nearly all of the gas vesicles were intact and ready for use in subsequent applications.

### Hexapeptide Characterization

In one embodiment, the IK₆ amphiphilic peptide (SEQ ID No. 1) has a hydrophilic head group at the C terminus followed by a series of hydrophobic residues, increasing in hydrophobicity to the N terminus at the tail end of the peptide.

In one embodiment, the IK₆ amphiphilic peptide may be optionally connected to an N-terminal protecting group, preferably acetylated, and may be amidated o by a C-terminal protecting group, resulting in a sequence of Ac-ILVAGK-NH₂ (SEQ ID No. 1).

In one embodiment, the IK₆ peptide (SEQ ID No. 1) readily forms a hydrogel in PBS.

In one embodiment, the IK₆ peptide (SEQ ID No. 1) has rapid self-assembly capability and is ease of use.

In one embodiment, the IK₆ peptide (SEQ ID No. 1) has high degree of biocompatibility.

In one embodiment, the IK₆ peptide (SEQ ID No. 1) has demonstrated excellent gelling properties as it gelated quickly enough to ensure the structural integrity of the construct and printed smoothly to avoid clumping or inconsistencies. Therefore, the IK₆ peptide (SEQ ID No. 1) is used as bioink, when mixed with cellularized components, in one embodiment.

It has been previously reported that self-assembling ultrashort tetrapeptide forms fibrous peptide network formation, characterizing the structures with circular dichroism (CD) and X-ray diffraction (XRD)⁴⁴. In one embodiment, the peptide IK₆ (SEQ ID No. 1) forms similar peptide networks as self-assembling ultrashort tetrapeptide compounds.

In one embodiment, the network formed by IK₆ peptide (SEQ ID No. 1) was characterized with scanning electron microscopy (SEM) after printing with a printing system. FIG. 5 shows the morphological characterization of the printed IK₆ peptide (SEQ ID No. 1) scaffold by SEM. The condensed fibers of IK₆ hydrogels shown in FIG. 5 was at a concentration of 16 mM. The In FIG. 5, the left panel was obtained at 200,000× magnification, while the right panel was obtained at 50,000× magnification.

In one embodiment, the IK₆ hydrogel's formation of fibrous network is throughout the construct.

In one embodiment, the morphological characteristics of hydrogel construct prepared with 3D printing system are similar to those in hydrogel samples prepared manually. The samples prepared for imaging were at a concentration of approximately 16 mM.

In one embodiment, this concentration of IK₆ peptide (SEQ ID No. 1) used with the cells and GVNPs was also 16 mM.

In one embodiment, the printing of at IK₆ hydrogels at 16 mM occurred smoothly and consistently.

### Effect of GVNPs on Cell Viability in 3D Construct

Halobacterium sp. NRC-1 gas vesicle cytotoxicity is assessed through the 2D culture of HEK cells²². In one embodiment, the cell viability is nearly 100%, at the concentrations of GVNPs up to 500 μg/mL²²

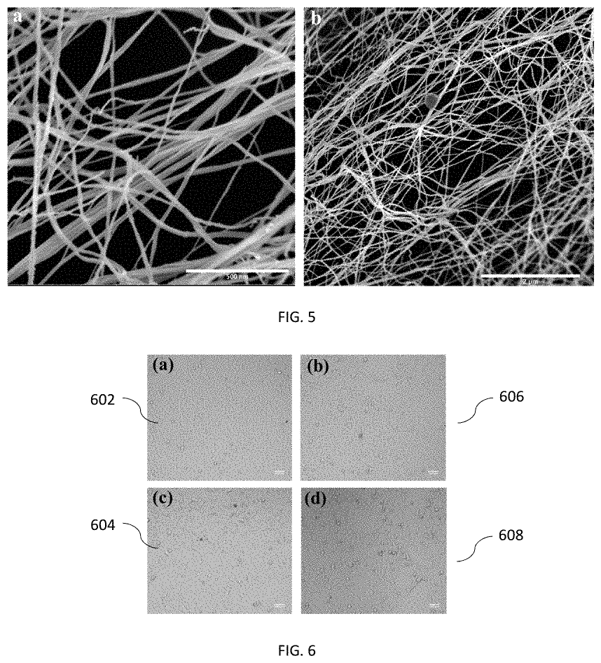

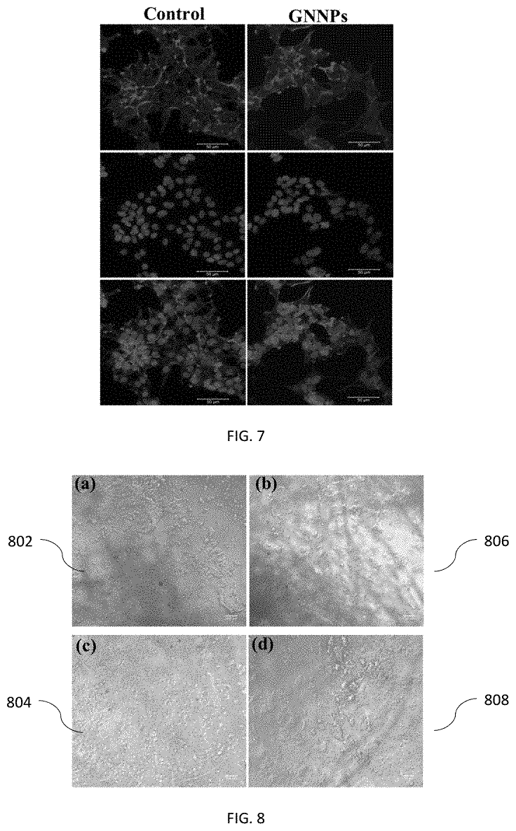

In one embodiment, cytotoxicity of gas vesicles expressed in Haloferax volcanii was assessed by testing cell growth and proliferation in 2D culture and 3D culture with varying concentrations of GVNPs. FIGS. 6 and 7 show the cell proliferation of HEK cells cultured in 2D culture together with GVNPs.

FIG. 6 shows the bright-field microscopic images characterizing the HEK cells in 2D after one day of culture, with different concentrations of GVNPs, including 0 μg/mL (control) (602), 250 μg/mL (606), 500 μg/mL (604), and 750 μg/mL (608).

FIG. 7 shows cell morphology of HEK cells in 2D with fluorescent staining. In FIG. 7, HEK cells cultured without GVNPs is shown in the left column, while HEK cells cultured with 750 μg/mL GVNPs is shown in the right column. Also in FIG. 7, actin is shown in red, while nucleus are shown in blue.

FIG. 8 shows he bright-field microscopic images characterizing the HEK cells in 3D after one day of culture, with different concentrations of GVNPs, including 0 μg/mL (control) (802), 250 μg/mL (806), 500 μg/mL (804), and 750 μg/mL (808).

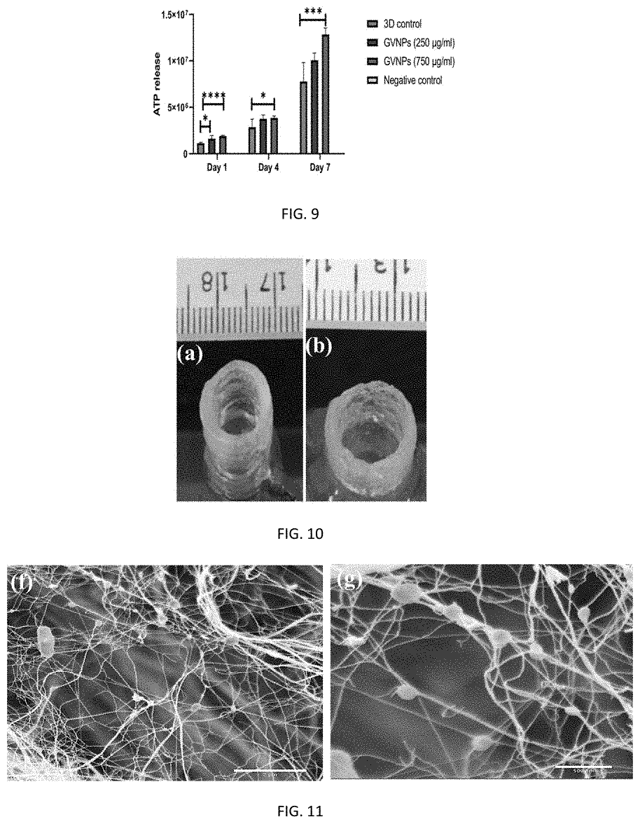

In one embodiment, cell proliferation is quantified with the CellTiter-Glo© Assay. FIG. 9 shows the results of the CellTiter-Glo© Assay. The HEK cells are cultured with GVNPs at the concentration of 250 μg/mL and 750 μg/mL. As shown in FIG. 9, an increase in cell activity is observed from day 1 to day 7. When compared to the 3D and negative control groups, the cells cultured with GVNPs show significantly increased proliferation, as also shown in FIG. 9. With regard to the 3D control in FIG. 9, the HEK cells are cultured in DMEM medium without GVNPs. Furthermore, cell proliferation was observed to increase with increasing concentrations of GVNPs. FIG. 9 shows a mean value±SD (standard deviation), with a sample size of 6. In FIG. 9, * indicates p<0.05, while *** indicates p<0.001 The differences in cell proliferation was compared between the 3D control and cells cultured with GVNPs using student's t-test and the p values are summarized below:

| | Day 1 | Day 4 | Day 7 |
|---|---|---|---|
| 250 μg/mL | 0.006 | 0.04 | 0.03 |
| 750 μg/mL | 0.1 × 10⁻⁴ | 0.02 | 0.0002 |

### 3D Bioprinting Process

In one embodiment, various shapes are printed using IK₆ peptide (SEQ ID No. 1) to optimize printing parameters, including peptide and PBS flow rates.

In one embodiment, the rapid gelation of the IK₆ peptide (SEQ ID No. 1) facilitate smooth and consistent extrusion of the bioink, thus achieving a more refined and stable construct of 10 mm in height. The construct of 10 mm height formed with IK₆ peptide (SEQ ID No. 1) is shown in FIG. 10. In FIG. 10, the left panel shows cylinder printed with IK₆ peptide (SEQ ID No. 1) on day 1, while the right panel shows the same construct after 8 weeks.

In one embodiment, IK₆ peptide has high degree of bioprintability, structure fidelity, and cell viability⁴⁵.

In one embodiment, the stability of the bioprinted construct with IK₆ peptide (SEQ ID No. 1) is high enough to maintain high degree of construct fidelity during the incubation period of several weeks.

In one embodiment, the construct maintained structure and hold shape, after 8 weeks, thus confirming the high structure fidelity of the IK₆ peptide (SEQ ID No. 1).

In one embodiment, the gas vesicles are suitable for bioprinting.

In one embodiment, the optimal printing parameters of bioprinting using gas vesicles are determined.

Previous studies find that gas vesicles are relatively weak and their strength varies depending on the organism and strain from which they are derived^{19, 21}. However, in one embodiment, the gas vesicles may withstand the stress of the 3D printing process.

In one embodiment, the morphology of the printed gas vesicles is c compared to those that were not printed. Analysis of these images via SEM imaging, confirming that the gas vesicles remained intact and maintained their structural integrity throughout the printing process. The SEM images are shown in FIG. 11. In FIG. 11, the left panel shows photo of printed peptide scaffold with GVNPs obtained at 35,000× magnification, while the right panel shows that obtained at 100,000× magnification.

In one embodiment, the addition of gas vesicles can improve oxygen availability to cells within the center of printed constructs.

In one embodiment, optimal gas vesicle distribution within the samples is determined.

In one embodiment, gas vesicle distributes homogenously throughout the construct.

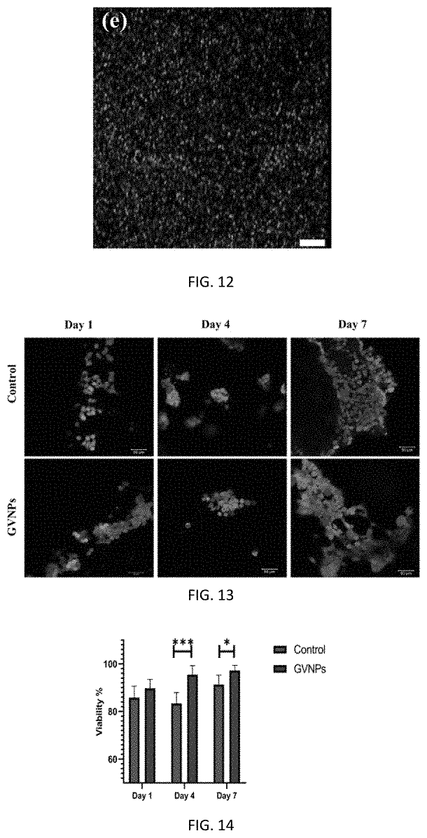

In one embodiment, fluorescent gas vesicle particles engineered with sfGFP is created and used to determine the homogenous distribution of gas vesicle throughout the construct. FIG. 12 shows a confocal image of these samples printed with sfGFP engineered gas vesicles confirming a homogenous distribution of gas vesicles throughout the printed sample. In FIG. 12, the scale bar is 2 μm.

### Influence of GVNPs on the Viability of Bioprinted Cells

In one embodiment, the HEK 293 cells are 3D printed, and the cell viability with the presence of GVNPs is increased at 3 different time points, including 24 hours, 4 days and 7 days, compared to cells cultured without GVNPs.

FIG. 13 shows a live/dead cell staining image, comparing cell viability of HEK 293 cells s cultured with GVNPs or without GVNPs (control).

FIG. 14 shows the quantification of cell viability, indicating the presence of GVNPs increaded cell viability. The cell viability data are summarized below, with the data expressed as mean value±SD. The differences of cell viability on day 4 and day 7 between control and those grown with GVNPs are analyzed using student's t test, with the p value also summarized in the table below.

| | 24 hours | Day 4 | Day 7 |
|---|---|---|---|
| control | 85.8 ± 4.8% | 83.4 ± 4.6% | 91.3 ± 3.9% |
| GVNPs | 89.7 ± 3.7% | 92.6 ± 3.7% | 96.4 ± 2.1% |
| p value | | 0.0005 | 0.009 |

### Morphological Study of Bioprinted Cells

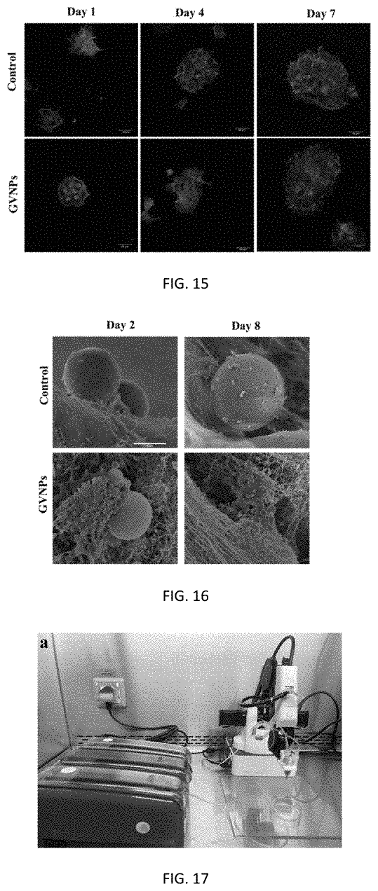

In one embodiment, cell aggregates form within gels after printing. In one embodiment, the cells proliferate within gels after printing The actin cytoskeletons and nuclei of these cell aggregates were stained, as shown in FIG. 15. In FIG. 15 the growth of the cell aggregates was tracked for 7 days. The growing size of the cellular aggregates over time is evidence for cell proliferation. In FIG. 15, the nucleus are shown in blue, F-actin in red and the scale bar is 50 μm.

In one embodiment, the GVNPs in the gel construct after printing are visualized using microscopy imaging. These images depicting the GVNPs in the environment surrounding the cells 2 and 8 days after printing are shown in FIG. 16. This is contrasted with the lack of gas vesicles present in the imaged control samples. In FIG. 16, the scale bar is 5 μm.

In one embodiment, a new plasmid construct allowing fast and high-yielding GVNPs production in Haloferax volcanii without the requirement of antibiotics is provided. In one embodiment, Haloferax volcanii is completely sequenced and has clean genetic background with respect to gas vesicle genes, making it a suitable and attractive organism of choice for expressing Halobacterium gas vesicle nanoparticles. H. volcanii is a nonpathogenic halophile that grows to high density in the presence of high concentrations of salt, which precludes contamination by less halophilic microorganisms^{42, 46}. In one embodiment, the S-layer cell walls of H. volcanii is fragile enough to be easily lysed upon addition of water, releasing cellular proteins and reducing the cost of protein purification^{47, 48}, which also makes it attractive feature for biotechnological and biomedical applications.

In one embodiment, the Superfolder Green Fluorescent Protein (sfGFP) is displayed on the GVNPs surface, which demonstrated the application of GVNPs as imaging tools and oxygen carriers as well as protein display and delivery vehicle.

In one embodiment, the protein expression system and purification process are straightforward and efficient enough to produce a substantial number of gas vesicles that can be scaled.

In one embodiment, the GVNPs has a spindle or cylindrical shape, which increases volume to surface area ratio. The increased volume to surface area ratio allows improved gas exchange and nutrient diffusion.

In one embodiment, the diameter/width of the gas vesicles ranging from 60-200 nm is also ideal for biomedical applications. GVNPs are small enough to fit within the pores of the fibrous extracellular matrix of hydrogels while still too large to cross the blood-brain barrier and cell membrane⁴⁹.

The biocompatibility of Halobacterium sp. NRC-1 gas vesicles in 2D cell culture was studied²². In one embodiment, gas vesicles expressed in Haloferax volcanii has high biocompatibility, as confirmed by 3D cell culture of HEK 293 cells with various concentrations of GVNPs. In one embodiment, the gas vesicles have no associated toxicity, as indicated by the qualitative and quantitative assessment concerning cell growth, morphology, and proliferation.

In one embodiment, the bioink and gas vesicles survives the stress associated with the printing process. In one embodiment, comparison of the imaged printed and manually prepared peptide bioink samples confirmed that the peptide maintained its ability to self-assemble into helical fibers and form supramolecular structures, despite the shear stress imposed by the printing.

In one embodiment, the versatility of the printing system shown in FIG. 17 is suitable for the use of ultrashort self-assembling peptide as bioink material and the incorporation of gas vesicles in the constructs.

In one embodiment, nearly all of the 3D printed gas vesicles remained intact, thereby providing further evidence of the strength of halophilic gas vesicles and suggesting potential applications of gas vesicles for biomedical applications.

In one embodiment, recombinant GVNPs are stable for several months at room temperature and at elevated temperatures of around 50° C., with little to no degradation²⁷.

In one embodiment, these protein-based nanostructures can promote the growth of printed cells, which is crucial to preserve cell viability, morphology, and function after printing ⁵⁴⁻⁵⁷. Previous studies have reported that the stress induced by the printing process may harm the cells^{57, 58}. However, the extent of this cellular damage may differ according to cell type and the printing procedure.

In one embodiment, in the case of 3D bioprinter using the configuration shown in FIG. 17, the printing procedure could be modified by adjusting syringe pump flow rates, inner diameters of the nozzle, printing speed, and layer height.

In one embodiment, the 3D bioprinting parameters are optimized for HEK 293 cells and incorporated the GVNPs into the 3D construct, which were mitigated such that cell viability, proliferation, and morphology were improved.

The notion that cell growth and death are very strongly affected by the extracellular environment has been well documented in the literature⁵⁹. The extracellular matrix (ECM) is crucial to tissue engineering, as it dictates cellular morphology and guides the connections between cells and the site of interactions. Together, the ECM architecture and its composition affect cell growth, connection, differentiation, and adhesion. In one embodiment, the GVNPs were able to incorporate within the peptide fibers. The gas vesicles were also able to stay within the extracellular environment for at least 8 days, increase the porosity of the gel constructs, and thereby allow more medium and nutrients to penetrate the center.

In one embodiment, the GVNPs are able to influence the ECM without directly affecting the morphology of the HEK293 cells, as shown in FIGS. 13, 15 and 16.

In one embodiment, the cells printed with GVNPs show better viability than the cells printed without GVNPs. As the GVNPs constitute the thin protein-membrane and the gases inside and are permeable to gases in the environment, the effect is likely due to the gas permeability of the protein shell. One way, through which this can occur, is by promoting the availability of oxygen to the cells, as the oxygen is allowed to diffuse from within the gas vesicles to the surrounding environment. In one embodiment, the increased oxygen availability may render the environment more similar to favorable in vivo conditions, thereby promoting cell survival. In another embodiment, nearby GVNPs enable cells to clear waste or other species within the environment more rapidly. Byproducts of cellular respiration include CO₂. Therefore, the permeability of the GVNP shell coupled with the concentration gradient of the different gases in the system may have promoted the diffusion of CO₂ away from the cells.

In one embodiment, Gas vesicles are excellent materials for applications in medicine, due to their aforementioned advantages over other nanostructures. In another embodiment, these gas-filled stable protein nanostructures are uniquely well suited for bioengineering.

In one embodiment, the use of genetically encoded air-filled gas vesicles for 3D bioprinting is demonstrated for the first time. In one embodiment, the gas vesicles does not harm cell proliferation, despite its bacterial origination. In one embodiment, cell viability is promoted by the gas vesicles.

In one embodiment, the additional oxygen support is beneficial. This extra support is beneficial for the cells that are encapsulated within the center of the 3D printed construct and thus due to the diffusion barrier less accessible to nutrients. Thus, air-filled GVNPs that are printed together with living cells can counterbalance stress factors which the cells are experiencing during and after 3D printing.

In one embodiment, promoting viability of bioprinted cells with the addition of GVNPs facilitates the 3D printing of large tissues and organs.

In one embodiment, using gas vesicles in tissue engineering applications enhances the proliferation of mammalian cells in 3D cultures. In one embodiment, the gas vesicles can withstand the stress associated with the bioprinting process and positively influence cell viability suggest that this method can promote the survival of cells within 3D printed constructs. In one embodiment, the gas vesicles can be used as a suitable and easy-to-prepare tool to promote cell viability during tissue construct formation and can serve as an oxygen place holder during nascent vascularization approaches.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

### Materials

The self-assembling peptide IK₆ (Ac-ILVAGK-NH₂) (SEQ ID No. 1) was custom synthesized by Bachem® AG (Budendorf, Switzerland). Human embryonic kidney cells (HEK293) were purchased from (ATCC®, USA). Cells were cultured in medium Dulbecco's modified Eagle's medium-high glucose DMEM-HG (Gibco Thermo Fisher Scientific®, USA). T175 or T75 cell culture flasks and 96 and 48 well-plates were purchased from Corning, USA. Halobacterium sp. NRC-1 was obtained from Carolina Biological Supply (Burlington, N.C., USA) and cultured in CM⁺ medium containing 4.3M NaCl and trace metals at 42° C. with shaking as previously described 37. Haloferax volcanii H1895 and its corresponding vector pTA963 were kindly provided by Dr. Thorsten Allers (Institute of Genetics, School of Biology, University of Nottingham, Queen's Medical Centre, Nottingham, UK). Haloferax volcanii and derivatives were cultured in the Hv-YPC medium at 45° C. with shaking as previously described 3, 39. For solid media, 2% (w/v) agar was added. The CellTiter-Glo® luminescent 3D cell viability assay kit, LIVE/DEAD® Viability/Cytotoxicity kit and Actin Cytoskeleton/Focal Adhesion Staining kit were purchased from Promega, USA, Life Technologies™, USA, and Sigma-Aldrich®, USA, respectively.

### Example 2

Engineering and Expression of Gas Vesicles in Haloferax volcanii

sfGFP synthetic gene (IDT®, Leuven, Belgium) was codon-optimized using the java codon adaptation online tool JCat for Halobacterium sp. (strain NRC-1/ATCC 700922/JCM 11081)⁴⁰. The gas vesicle operon from Halobacterium sp. NRC-1 was amplified from the genome by Polymerase Chain Reaction (PCR) and cloned with sfGFP via FspAI-HpaI and HpaI-BamHI using the Gibson Assembly Cloning Kit into plasmid vector pTA963 to generate the pTA963_sfGFP_GVNPs expression plasmid (Table 1). The construct was validated by restriction digestion using FspAI, HpaI, and BamHI, PCR amplification, and DNA sequencing. Gas vesicles containing the vector were transformed into Haloferax volcanii H1895 using the PEG/EDTA method⁴¹

| Primer | 5′-3′ sequence |
|---|---|
| pTA.1 | GGACCTATTGCGCATATGCACCACCACCACCAC |
| (SEQ ID No. 2) | CACATGCGCATAATTCAATCGATACGAGTCCCG |
| pTA.2 | AATGCGATGGTCCAGAGGTGCGGCCGCTCTAGA |
| (SEQ ID No. 3) | ACTAGTGGATCCGATCTGTGAGTGTACACCCC |
| HpaI-BamHI | TGTCTCTTCTTCCTCGTTAACGGTACCGGCGGA |
| (SEQ ID No. 4) | TTCTCC |
| FspAI-HpaI | GCGGAGAATCCGCCGGTACCGTTAACGAGGAAG |
| (SEQ ID No. 5) | AAGAGACAGAGCC |

### Example 3

### Culturing and Gas Vesicle Preparation

The processes for producing and culturing gas vesicles were performed as previously described ^{19, 22, 42}. Briefly, H. volcanii lawns or floating cells were lysed osmotically with PBS solution [137 mM NaCl, 2.7 mM KCl, 10 mM sodium phosphate dibasic, and 2 mM potassium phosphate monobasic (pH 7.4)] containing 10 mM MgSO₄ and 20 μg/mL of DNase I (Sigma-Aldrich®, USA). The cell lysate suspension was incubated for 1 hour at 37° C. before overnight centrifugation at 60 g in a swinging bucket rotor in an Allegra® X-15R centrifuge (Beckman Coulter®, CA, USA) to accelerate floatation of the gas vesicles. Intact gas vesicles were collected and re-suspended in PBS solution, then floated by overnight centrifugation and harvested again. This floatation procedure was repeated until a white, milky suspension of gas vesicles was obtained. Gas vesicle concentration was quantified via NanoDrop® 2000 spectrophotometer (Thermo Scientific®, Waltham, Mass., USA) by measuring a small sample of gas vesicles broken by sonicating for several minutes.

### Example 4

### Hydrogel Preparation

The ultrashort peptide IK₆ (Ac-ILVAGK-NH₂) (SEQ ID No. 1) used in this study was synthesized by Bachem® AG (Budendorf, Switzerland) using solid-phase peptide synthesis and purified to above 95% via HPLC. Amino acid and peptide content analysis were performed. The lyophilized peptide powders were first dissolved in Milli-Q® water and mixed by vortexing for 30 seconds to obtain a homogenous solution. Then, 10× phosphate-buffered saline (PBS) was added to the peptide solution for a final concentration of 1×. Gelation occurred within a few minutes at 8 mg/mL peptide concentration.

### Example 5

### Cell Culture in 2D

HEK293 cells were purchased from ATCC® (USA). Cells were cultured in Dulbecco's modified Eagle's medium-high glucose (DMEM-HG), supplemented with 10% (v/v) fetal bovine serum (FBS) and 1% penicillin/streptomycin (PS, Gibco®) at 37° C. with 5% CO₂. The cells were subcultured with trypsin at approximately 80% confluence. The culture media was changed every 2-3 days. Cells at passages 6-8 were encapsulated for 3D culture and monolayer culture.

### Example 6

### Cell Culture in 3D

HEK293 cells were cultured in 75 T flasks and incubated in a CO₂ incubator maintained at 37° C. with 5% CO₂. Culture media was replaced every 48 hours until the cells reached 80% confluency. Confluent cells were subcultured, and cells at passages 6-8 were used for the study. For the 3D culture, the peptide was sterilized by exposure to UV light for 30 minutes. 10,000 cells in 2× PBS were mixed at a 1:1 ratio with peptide solution and used to prepare 100 μl of 3D construct in a 96 well-plate without the addition of GVNPs to serve as a control. Different concentrations of GVNPs were obtained by mixing with PBS before adding them to form 3D samples. This allowed for the evaluation of the GVNPs effect on cell proliferation.

### Example 7

### 3D Bioprinting

16 mM of IK₆ peptide (SEQ ID No. 1) was diluted in 1 mL of MilliQ® water, mixed well, and sonicated to assure a homogenous solution. Eight million cells were suspended in 1× PBS without GVNPs (control). When printing with GVNPs, the cells were mixed with a 1× PBS solution containing GVNPs at a 300 μg/ml concentration.

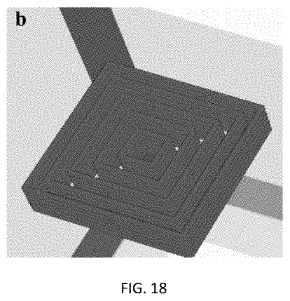

A custom-designed 3D bioprinter along with commercial microfluidic pumps was set up, and a homemade dual coaxial nozzle was used for bioink extrusion^{32, 33, 43}. Structures were printed in the shape of a rectangular prism with a length, width, and height of 10 mm, 10 mm, and 1.5 mm, respectively. Illustrated figure of the printed structure along with the printer setup is shown in FIG. 17. A preview of the gcode file of the printed structure with dimensions measuring 10 mm×10 mm×1.5 mm is shown in FIG. 18. To facilitate imaging, the structure was printed onto an 18×18 mm glass coverslip. The glass coverslip was then placed on top of a heat-bed set to 40° C. to create a suitable temperature environment for the cells.

Three syringe pumps were set up to dispense homogenous gel and extrude peptide bioink through the nozzle. The first syringe pump was loaded with peptide solution and set to a flow rate of 55 μl/min. The second pump was loaded with 7× PBS and set to a flow rate of 20 μl/min. The third pump was loaded with the cells and set to a flow rate of 20 μl/min. Three samples of the 4-layer rectangular prism were printed for each condition with a height of 1.5 mm per sample to facilitate imaging. The same procedure was conducted for samples without gas vesicles to serve as controls.

### Example 8

### 3D Cell Proliferation Assay

The CellTiter-Glo® luminescent 3D cell viability assay was used to assess cell proliferation in 3D hydrogels by measuring ATP production. At each time point, the kit was equilibrated at room temperature for approximately 30 minutes. CellTiter-Glo® Reagent equal to the volume of cell culture medium present in each well was added. The contents were mixed for 5 minutes to digest the hydrogels and then incubated for 30 minutes. After incubation, the luminescence was recorded using a plate reader (PHERAstar® FS, Germany).

### Example 9

### Live/Dead Staining

HEK293 cells were seeded into peptide according to the protocol described in Example 6 above. After one, three, and seven days of incubation, the media was removed and replaced with PBS solution containing staining solution approximately 2 mM calcein AM and 4 mM ethidium homodimer-1 (staining solution) prior to incubation for 40 minutes. Before imaging, the staining solution was removed, and fresh PBS was added. Stained cells were imaged under an inverted confocal microscope (Zeiss® LSM 710 Inverted Confocal Microscope, Germany). The percentage of living cells was obtained through analysis with ImageJ.

### Example 10

### Cytoskeletal Staining and Imaging

Immunostaining was performed at each culture time point. In brief, the cells were fixed with 4% (v/v) paraformaldehyde solution for 30 minutes and incubated in a cold cytoskeleton buffer (3 mM MgCl₂, 300 mM sucrose, and 0.5%, v/v Triton X-100 in PBS solution) for 5 minutes to permeabilize the cell membranes. The permeabilized cells were then incubated in blocking buffer solution (5% FBS, 0.1%, v/v Tween-20, and 0.02%, w/v sodium azide in PBS) for 30 minutes at 37° C. For F-Actin, anti-mouse IgG (whole molecule)-FITC and rhodamine-phalloidin (1:300) were added to the cells for 1 hour. Further, the cells were incubated in DAPI for five minutes to counterstain the nucleus. The fluorescent dye-treated cells were observed and imaged using a laser scanning confocal microscope (Zeiss® LSM 710 Inverted Confocal).

### Example 11

### Scanning Electron Microscopy

The printed samples were characterized using Scanning Electron Microscopy (SEM) to visualize the morphology of the peptide bioink and gas vesicles in printed samples⁴³. Samples were printed on 18×18 mm glass coverslips and given time to solidify before dehydration by gradual immersion in increasing concentrations of 20%, 40%, 60%, 80%, and 100% (v/v) ethanol solutions for 5 min in each solution. Further dehydration in 100% ethanol solution was done by changing the absolute ethanol solution with a fresh ethanol solution twice for 5 min each, followed by a third time for 2 hours. Dehydrated samples were subsequently placed into the critical point dryer (Sorvall Critical Point Drying System) for evaporation before being mounted onto SEM aluminum pin stubs with double-stick conductive carbon tape and a final sputter coating of 10 nm of iridium. Images were taken with FEI Magellan XHR SEM. This was done with a TLD detector, and imaging parameters included a current of 50 pA, a high voltage of 3.00 kV, and a working distance of 4.0 m. Biological peptide hydrogel coating cells were fixed with 2.5% (v/v) glutaraldehyde (diluted from 25%) in water overnight at 4° C., the post-fixation was done by 1% (w/v) osmium tetroxide in 0.1 M PBS for one hour in the dark followed by washing 3 times by ddH₂O. This was followed by serial dehydration with 10 mL of H₂O (twice), 25% ethanol, 50% ethanol, 75% ethanol, 80% ethanol, 90% ethanol, and 100% (v/v) ethanol (twice). Samples in ethanol were then critically point dried using liquid CO₂ (Sorvall Critical Point Drying System).

### Example 12

### Statistical Analysis

All experimental approaches were executed in triplicates to allow for statistical testing. Results are represented as mean±standard deviation, n≥3. The differences observed in HEK293 cell behavior for conditions with and without GVNPs were compared. Statistical analysis was performed by a student's t-test, and values with p<0.05 were considered statistically significant.

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. Cui H, Nowicki M, Fisher J P and Zhang L G 2017 3D Bioprinting for Organ Regeneration. Adv. Healthc. Mater. 6 1601118.
2. Khademhosseini A and Langer R A 2016 decade of progress in tissue engineering. Nat. Protoc. 11 1775.
3. Bishop E S, Mostafa S, Pakvasa M, Luu H H, Lee M J, Wolf J M, Ameer G A, He T C and Reid R R 2017 3-D bioprinting technologies in tissue engineering and regenerative medicine: Current and future trends. Genes & Diseases 4 185.
4. Murphy S V and Atala A 2014 3D bioprinting of tissues and organs. Nat. Biotechnol. 32 773.
5. Seol Y J, Kang H W, Lee S J, Atala A and Yoo J J 2014 Bioprinting technology and its applications. Eur. J. Cardiothorac. Surg. 46 342.
6. Ravnic D J, Leberfinger A N, Koduru S V, Hospodiuk M, Moncal K K, Datta P, Dey M, Rizk E and Ozbolat I T 2017 Transplantation of Bioprinted Tissues and Organs: Technical and Clinical Challenges and Future Perspectives. Ann. Surg. 266 48.
7. Hauser C A E and Zhang S G 2010 Designer self-assembling peptide nanofiber biological materials. Chem. Soc. Rev. 39 2780.
8. Chen C, Bang S, Cho Y, Lee S, Lee I, Zhang S and Noh I 2016 Research trends in biomimetic medical materials for tissue engineering: 3D bioprinting, surface modification, nano/micro-technology and clinical aspects in tissue engineering of cartilage and bone. Biomater. Res. 20 10.
9. Visconti R P, Kasyanov V, Gentile C, Zhang J, Markwald R R and Mironov V 2010 Toward organ printing: engineering an intra-organ branched vascular tree. Expert Opin. Biol. Ther. 10 409-420
10. Kolesky D B, Homan K A, Skylar-Scott M A and Lewis J A 2016 Three-dimensional bioprinting of thick vascularized tissues. PNAS 113 3179.
11. Zhang B, Luo Y, Ma L, Gao L, Li Y, Xue Q, Yang H and Cui Z 2018 3D bioprinting: an emerging technology full of opportunities and challenges. Bio-Design and Manufacturing 1 2.
12. Zhang Y S, Yue K, Aleman J, Mollazadeh-Moghaddam K, Bakht S M, Yang J, Jia W, Dell'Erba V, Assawes P, Shin S R, Dokmeci M R, Oklu R and Khademhosseini A 2017 3D Bioprinting for Tissue and Organ Fabrication. Ann. Biomed. Eng. 45 148.
13. Suvamapathaki S, Wu X, Lantigua D, Nguyen M A and Camci-Unal G Breathing life into engineered tissues using oxygen-releasing biomaterials. NPG Asia Mater. 11 65.
14. Erdem A, Darabi M A, Nasiri R, Sangabathuni S, Ertas Y N, Alem H, Hosseini V, Shamloo A, Nasr A S and Ahadian S 2020 3D Bioprinting of Oxygenated Cell-Laden Gelatin Methacryloyl Constructs. Adv. Healthc. Mater. 9 1901794.
15. Pedraza E, Coronel M M, Fraker C A, Ricordi C and Stabler C L 2012 Preventing hypoxia-induced cell death in beta cells and islets via hydrolytically activated, oxygen-generating biomaterials. PNAS 109 4245.
16. McQuilling J P, Sittadjody S, Pendergraft S, Farney A C and Opara E C 2017 Applications of particulate oxygen-generating substances (POGS) in the bioartificial pancreas. Biomater. Sci. 5 2437.
17. Ward K R, Huvard G S, McHugh M, Mallepally R R and Imbruce R 2013 Chemical oxygen generation. Respir. Care 58 184.
18. Pfeifer F 2015 Haloarchaea and the formation of gas vesicles. Life 5 385.
19. DasSarma S, Karan R, DasSarma P, Barnes S, Ekulona F and Smith B 2013 An improved genetic system for bioengineering buoyant gas vesicle nanoparticles from Haloarchaea. BMC Biotechnol. 13 112.
20. Kunth M, Lu G J, Witte C, Shapiro M G and Schroder L 2018 Protein Nanostructures Produce Self-Adjusting Hyperpolarized Magnetic Resonance Imaging Contrast through Physical Gas Partitioning. ACS Nano 12 10939.
21. Pfeifer F 2012 Distribution, formation and regulation of gas vesicles. Nat. Rev. Microbiol. 10 705.
22. Andar A U, Karan R, Pecher W T, DasSarma P, Hedrich W D, Stinchcomb A L and DasSarma S 2017 Microneedle-assisted skin permeation by nontoxic bioengineerable gas vesicle nanoparticles. Mol. Pharm. 14 953.
23. Lu G J, Chou L D, Malounda D, Patel A K, Welsbie D S, Chao D L, Ramalingam T and Shapiro M G 2020 Genetically Encodable Contrast Agents for Optical Coherence Tomography. ACS Nano 14 7823.
24. Szablowski J O, Bar-Zion A and Shapiro M G 2019 Achieving spatial and molecular specificity with ultrasound-targeted biomolecular nanotherapeutics. Acc. Chem. Res. 52 2427.
25. Lu G J, Farhadi A, Szablowski J O, Lee-Gosselin A, Barnes S R, Lakshmanan A, Bourdeau R W and Shapiro M G 2018 Acoustically modulated magnetic resonance imaging of gas-filled protein nanostructures. Nat. Mater. 17 456.
26. Lakshmanan A, Farhadi A, Nety S P, Lee-Gosselin A, Bourdeau R W, Maresca D and Shapiro M G 2016 Molecular engineering of acoustic protein nanostructures. ACS Nano 10 7314.
27. DasSarma P, Negi V D, Balakrishnan A, Karan R, Barnes S, Ekulona F, Chakravortty D and DasSarma S 2014 Haloarchaeal gas vesicle nanoparticles displaying Salmonella SopB antigen reduce bacterial burden when administered with live attenuated bacteria. Vaccine 32 4543.
28. Balakrishnan A, DasSarma P, Bhattacharjee O, Kim J M, DasSarma S and Chakravortty D 2016 Halobacterial nano vesicles displaying murine bactericidal permeability-increasing protein rescue mice from lethal endotoxic shock. Sci. Rep. 6 1.
29. Loo Y, Lakshmanan A, Ni M, Toh L L, Wang S and Hauser C A E 2015 Peptide Bioink: Self-Assembling Nanofibrous Scaffolds for Three-Dimensional Organotypic Cultures. Nano Letters 15 6919.
30. Sundaramurthi D, Rauf S and Hauser C 2016 3D bioprinting technology for regenerative medicine applications. Int. J. Bioprinting 2 19.
31. Loo Y and Hauser C A 2015 Bioprinting synthetic self-assembling peptide hydrogels for biomedical applications. Biomed. Mater. 11 014103.
32. Khan Z, Kahin K, Rauf S, Ramirez-Calderon G, Papagiannis N, Abdulmajid M and Hauser C A 2018 Optimization of a 3D bioprinting process using ultrashort peptide bioinks. Int. J Bioprinting 5 173.
33. Kahin K, Khan Z, Albagami M, Usman S, Bahnshal S, Alwazani H, Majid M, Rauf S and Hauser C 2019 In Development of a robotic 3D bioprinting and microfluidic pumping system for tissue and organ engineering, Proc. of SPIE, the International Society for Optics and Photonics, Microfluidics, BioMEMS and Medical Microsystems 12 108750Q1
34. Rauf S, Susapto H H, Kahin K, Alshehri S, Abdelrahman S, Lam J H, Asad S, Jadhav S, Sundaramurthi D, Gao X and Hauser C A 2020 Self-assembling tetrameric peptides allow in situ 3D bioprinting under physiological conditions. J. Mater. Chem. B. 9 1069.
35. Susapto H H, Alhattab D, Abdelrahman S, Khan Z, Alshehri S, Kahin K, Ge R, Moretti M, Emwas A H and Hauser C A E 2020 Ultrashort Peptide Bioinks Support Automated Printing of Large-Scale Constructs Assuring Long-Term Survival of Printed Tissue Constructs. Nano Lett. 21 2719.
36. Sundararajan A and Ju L-K 2006 Use of cyanobacterial gas vesicles as oxygen carriers in cell culture. Cytotechnol. 52 139.
37. DasSarma S, Fleischmann E and Rodriguez-Valera 1995 F Media for halophiles. Archaea: A Laboratory Manual, Robb F T, Place A R, Sowers K R, Schreier H J, DasSarma S, Fleischmann M E 1 225.
38. Strillinger E, Grotzinger S W, Allers T, Eppinger J and Weuster-Botz, D 2016 Production of halophilic proteins using Haloferax volcanii H1895 in a stirred-tank bioreactor. Appl. Microbiol. Biotechnol. 100 1183.
39. Grotzinger S W, Karan R, Strillinger E, Bader S, Frank A, Al Rowaihi I S, Akal A, Wackerow W, Archer J A, Rueping M, Weuster-Botz D, Groll M, Eppinger J and Arold S T 2018 Identification and Experimental Characterization of an Extremophilic Brine Pool Alcohol Dehydrogenase from Single Amplified Genomes. ACS Chem. Biol. 13 161.
40. Grote A, Hiller K, Scheer M, Munch R, Nortemann B, Hempel D C and Jahn D 2005 a novel tool to adapt codon usage of a target gene to its potential expression host. Nucleic Acids Res. 33 W526.
41. Dyall-Smith M 2008 The Halohandbook-Protocols for Haloarchaeal Genetics, Version 7.0. Bathurst, NSW, Australia: http://www.haloarchaea.com/resources/halohandbook/Halohandbook_2008_v7. pdf.
42. Grötzinger S W, Karan R, Strillinger E, Bader S, Frank A, Al Rowaihi I S, Akal A, Wackerow W, Archer J A and Rueping M 2018 Identification and experimental characterization of an extremophilic brine pool alcohol dehydrogenase from single amplified genomes. ACS Chem. Biol. 13 161.
43. Ghalayini S, Susapto H H, Hall S, Kahin K and Hauser C 2019 Preparation and printability of ultrashort self-assembling peptide nanoparticles. Int. J. Bioprint. 5 109.
44. Hauser C A E, Deng R, Mishra A, Loo Y, Khoe U, Zhuang F, Cheong D W, Accardo A, Sullivan M B, Riekel C, Ying J Y and Hauser U A 2011 Natural tri- to hexapeptides self-assemble in water to amyloid beta-type fiber aggregates by unexpected alpha-helical intermediate structures. Proc. Natl. Acad. Sci. USA 108 1361.
45. Alshehri S, Susapto H H and Hauser C A 2021 Scaffolds from Self-Assembling Tetrapeptides Support 3D Spreading, Osteogenic Differentiation, and Angiogenesis of Mesenchymal Stem Cells. Biomacromol. published online.
46. Gustavo Ramirez-Calderon, Hepi H S and Hauser C A E 2021 Delivery of Endothelial Cell-Laden Microgel Elicits Angiogenesis in Self-Assembling Ultrashort Peptide Hydrogels in Vitro. ACS Appl Mater Interfaces, accepted for publication.
47. Khan Z, Kahin K, Melle F, Alhattab D and Hauser C A E 2019 Assessing the Bioprintability of Self-Assembling Peptide Bioinks in Terms of Structure Fidelity and Cell Viability. Proc. of Ninth International Conference on Advances in Applied Science and Environmental Technology 8.
48. Akal A L, Karan R, Hohl A, Alam I, Vogler M, Grotzinger S W, Eppinger J and Rueping M 2019 A polyextremophilic alcohol dehydrogenase from the Atlantis II Deep Red Sea brine pool. FEBS Open Bio 9 194.
49. Vogler M, Karan R, Renn D, Vancea A, Vielberg M.-T, Grotzinger S W, DasSarma P, DasSarma S, Eppinger J and Groll M 2020 Crystal structure and active site engineering of a halophilic γ-carbonic anhydrase. Front. Microbiol. 11 742
50. Karan R, Mathew S, Muhammad R, Bautista D B, Vogler M, Eppinger J, Oliva R, Cavallo L, Arold S T and Rueping M 2020 Understanding high-salt and cold adaptation of a polyextremophilic enzyme. Microorganisms 8 1594
51. Silva I C S d, Delgado A B T, Silva P H V d and Costa V R X 2018 Focused ultrasound and Alzheimer's disease A systematic review. Dementia & Neuropsychologia 12 353.
52. Stuart E S, Morshed F, Sremac M and DasSarma S 2001 Antigen presentation using novel particulate organelles from halophilic archaea. J Biotechnol. 88 119.
53. DasSarma P, Negi V, Balakrishnan A, Kim J.-M, Karan R, Chakravortty D and DasSarma S 2015 Haloarchaeal gas vesicle nanoparticles displaying Salmonella antigens as a novel approach to vaccine development. Procedia in Vaccinol 9 16.
54. Sremac M and Stuart E S 2008 Recombinant gas vesicles from Halobacterium sp. displaying SIV peptides demonstrate biotechnology potential as a pathogen peptide delivery vehicle. BMC Biotechnol. 8 9.
55. Stuart E S, Morshed F, Sremac M and DasSarma S 2004 Cassette-based presentation of SIV epitopes with recombinant gas vesicles from halophilic archaea. J. Biotechnol. 114 225.
56. Nair K, Gandhi M, Khalil S, Yan K C, Marcolongo M, Barbee K and Sun W 2009 Characterization of cell viability during bioprinting processes. BTJ: Healthcare Nutrition Technology 4 1168.
57. Yu Y, Zhang Y, Martin J A and Ozbolat I T 2013 Evaluation of cell viability and functionality in vessel-like bioprintable cell-laden tubular channels. J Biomech. Eng. 135
58. Yan K C, Nair K and Sun W 2010 Three-dimensional multi-scale modelling and analysis of cell damage in cell-encapsulated alginate constructs. J. Biomech. 43 1031.
59. Jakab K, Norotte C, Marga F, Murphy K, Vunjak-Novakovic G and Forgacs G 2010 Tissue engineering by self-assembly and bio-printing of living cells. Biofabrication 2 022001.
60. Ferris C J, Gilmore K G and Wallace G G 2013 Biofabrication: an overview of the approaches used for printing of living cells. Appl. Microbiol. Biotechnol. 97 4243.
61. De Jong O G, Van Balkom, B W, Schiffelers R M, Bouten C V and Verhaar M C 2014 Extracellular vesicles: potential roles in regenerative medicine. Front. Immunol. 5 6083 686.
62. DasSarma S and DasSarma P 2015 Gas vesicle nanoparticles for antigen display. Vaccines 3 686.
63. Dutta S, DasSarma P, DasSarma S and Jarori G K 2015 Immunogenicity and protective potential of a Plasmodium spp. enolase peptide displayed on archaeal gas vesicle nanoparticles. Malar. J 14 406.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, products specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

1. A 3-dimensional construct comprising:
an ultrashort peptide scaffold;
at least one gas vesicle; and
at least one mammalian cell.

2. The 3-dimensional construct of claim 1, wherein the ultrashort peptide scaffold comprises at least one ultrashort peptide having a general formula selected from:
AₙBₘX,BₘAₙX,XAₙBₘ and XBₘAₙ
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of:
aspartic acid, glutamic acid, lysine, arginine, histidine, cysteine, serine, threonine, asparagine, and glutamine.

3. The 3-dimensional construct of claim 1, wherein the ultrashort peptide scaffold comprises an IK₆ peptide with the sequence of ILVAGK (SEQ ID No. 1),
wherein the IK₆ peptide is optionally connected to an N-terminal protecting group and a C-terminal protecting group.

4. The 3-dimensional construct of claim 3, wherein the N-terminal protecting group is an acetylated group and the C-terminal protecting group is an amidated group.

5. The 3-dimensional construct of claim 1, wherein the gas vesicle is homologously distributed in the 3D construct.

6. The 3-dimensional construct of claim 1, wherein the wall of the gas vesicle is gas, water and nutrient permeable.

7. The 3-dimensional construct of any claim 1, wherein the size of the gas vesicle is smaller than the pores of the ultrashort peptide scaffold.

8. The 3-dimensional construct of claim 1, wherein the size of the gas vesicle is too large to cross the blood-brain barrier and cell membrane of the mammalian cell.

9. The 3-dimensional construct of claim 1, wherein the diameter of the gas vesicle is 60-200 nm.

10. The 3-dimensional construct of claim 1, wherein the concentration of the gas vesicle is at least 250 μg/mL.

11. The 3-dimensional construct of claim 1, wherein the concentration of the gas vesicle is 250 μg/mL-750 μg/mL.

12. A method of high-yielding production of gas vesicle comprising:
amplifying a gas vesicle operon from the genome of a gas vesicle operon containing bacterium;
cloning the gas vesicle operon into an expression plasmid;
transforming the gas vesicle operon containing expression plasmid into Haloferax volcanii;
culturing the Haloferax volcanii transformed with the gas vesicle operon containing expression plasmid;
lysing the Haloferax volcanii transformed with the gas vesicle operon containing expression plasmid; and
collecting the gas vesicle.

13. The method of high-yielding production of gas vesicle recited in claim 12, wherein antibiotics are not used.

14. The method of high-yielding production of gas vesicle recited in claim 12, wherein a second operon is cloned into the expression plasmid.

15. The method of high-yielding production of gas vesicle recited in claim 12, wherein the second operon is for a sfGFP synthetic gene, wherein the sfGFP synthetic gene is optionally codon-optimized.

16. The method of high-yielding production of gas vesicle recited in claim 12, wherein the gas vesicle is collected by centrifugation and floatation

17. A method of creating 3-dimensional construct comprising:
dissolving at least one ultrashort peptide to form a peptide solution;
mixing mammalian cells with the peptide solution;
dissolving the gas vesicle to form a gas vesicle solution;
adding the gas vesicle solution to the mammalian cells containing peptide solution; and
building 3-dimensional construct using the peptide solution containing both gas vesicle and mammalian cells;
wherein the ultrashort peptide is dissolved in water or buffer solution.

18. The method of creating 3-dimensional construct in claim 17, wherein the ultrashort peptide is dissolved at a concentration from about 0.01 μg/ml to 100 mg/ml.

19. The method of creating 3-dimensional construct in claim 17, wherein peptide solution contains 16 mM of IK₆ peptide (SEQ ID No. 1).

20. The method of creating 3-dimensional construct in claim 17, wherein the final concentration of the gas vesicle in the 3-dimensional construct is at least 250 μg/mL.

21. The 3-dimensional construct of claim 17, wherein the final concentration of the gas vesicle in the 3-dimensional construct is 250 μg/mL-750 μg/mL.

22. The 3-dimensional construct of claim 17, wherein the final concentration of the gas vesicle in the 3-dimensional construct is 300 μg/mL.

23. The method of creating 3-dimensional construct in claim 17, wherein the number of cells in the 3-dimensional construct is at least 10,000.

24. The method of creating 3-dimensional construct in claim 17, wherein the 3-dimensional construct is built with a 3D bioprinter or manually.

25. A 3-dimensional construct comprising:
an ultrashort peptide scaffold, wherein the ultrashort peptide scaffold comprises at least one ultrashort peptide having a general formula selected from:
AₙBₘX,BₘAₙX,XAₙBₘ and XBₘAₙ
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine;
at least one mammalian cell; and
at least one gas vesicle, wherein the size of the gas vesicle is smaller than the pores of the ultrashort peptide scaffold but too large to cross the cell membrane of the mammalian cell.


SEQUENCE LISTING (5 sequences)

SEQ ID NO: 1 - protein, 6 aa, Artificial Sequence
  Synthetic IK6
ILVAGK

SEQ ID NO: 2 - DNA, 66 bp, Artificial Sequence
  Synthetic pTA.1
ggacctattgcgcatatgcaccaccaccaccaccacatgcgcataattcaatcgatacgagtcccg

SEQ ID NO: 3 - DNA, 65 bp, Artificial Sequence
  Synthetic pTA.2
aatgcgatggtccagaggtgcggccgctctagaactagtggatccgatctgtgagtgtacacccc

SEQ ID NO: 4 - DNA, 39 bp, Artificial Sequence
  Synthetic HpaI-BamHI
tgtctcttcttcctcgttaacggtaccggcggattctcc

SEQ ID NO: 5 - DNA, 46 bp, Artificial Sequence
  Synthetic FspAI-HpaI
gcggagaatccgccggtaccgttaacgaggaagaagagacagagcc